(19) United States
(12) Patent Application Publication
(10) Pub. No.: US 2024/0165418 A1
(43) Pub. Date: May 23, 2024

(54) CHARGING AND SPECIFYING A CAPACITOR FOR STORING CHARGE IN A WEARABLE CARDIAC DEFIBRILLATOR (WCD)

(71) Applicant: West Affum Holdings DAC, Dublin (IE)

(72) Inventors: Kenneth F. Cowan, Everett, WA (US); David P. Finch, Bothell, WA (US); Philip D. Foshee, JR., Woodinville, WA (US)

(73) Assignee: West Affum Holdings DAC, Dublin (IE)

(21) Appl. No.: 18/450,183

(22) Filed: Aug. 15, 2023

Related U.S. Application Data
(60) Provisional application No. 63/426,094, filed on Nov. 17, 2022.

(51) Int. Cl. A61N 1/39

(52) U.S. Cl. CPC A61N 1/3987; A61N 1/3904; A61N 1/3937; A61N 1/3975; A61N 1/3993

(57) ABSTRACT

Apparatus and methods for charging and specifying a capacitor for storing charge in a wearable cardioverter defibrillator (“WCD”). In one aspect the WCD circuitry includes a power source such as a battery coupled to a charger that provides charge energy to an energy storage module including an energy storage capacitor having a rated voltage. Control circuitry is configured to implement a multi-stage charging scheme under which the energy storage capacitor is charged to a first intermediate voltage below its rated voltage during a first charging stage and charged to a second voltage above the rated voltage for a limited time during a second charging stage. In response to detection of potential shockable rhythms the capacitor is charged to an intermediate voltage, while upon confirmation of shockable rhythms the capacitor may be charged to the second voltage, which is then used to deliver a shock to a patient.

### CROSS-REFERENCE(S) TO RELATED APPLICATION(S)

This patent application claims priority from U.S. provisional patent application Serial No. 63/426,094 filed Nov. 17, 2022, which is incorporated herein in its entirety for all purposes.

### NOTICE OF MATERIALS SUBJECT TO COPYRIGHT PROTECTION

Portions of the material in this patent document are subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, and for patent-related purposes only, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

A wearable medical system (“WMS”) is an advanced form of a medical system. A WMS typically includes one or more wearable components that a patient can wear or carry, and possibly other components that can be portable, or stationary such as base station and/or an electric charger. The WMS may also include one or more associated software packages, such as software applications (“apps”), which can be hosted by the wearable component, and/or by a mobile device, and/or by a remote computer system that is accessible via a communications network such as the internet, and so on.

A WMS typically includes one or more sensors that can sense when a parameter of the patient is problematic, and cause the WMS to initiate an appropriate action. The appropriate action could be for the WMS to communicate with the patient or even with a bystander, to transmit an alert to a remotely located clinician, and to even administer treatment or therapy to the patient by itself. The one or more sensors may sense more than one parameter of the patient. The multiple parameters may be used for determining whether or not to administer the treatment or therapy, or be suitable for detecting different problems and/or for administering respectively different treatments or therapies to the patient.

A WMS may also include the appropriate components for implementing a wearable cardioverter defibrillator (“WCD”), a pacemaker, and so on. Such a WMS can be for patients who have an increased risk of sudden cardiac arrest (“SCA”). In particular, when people suffer from some types of heart arrhythmias, the result may be that blood flow to various parts of the body is reduced. Some arrhythmias may result in SCA, which can lead to death very quickly, unless treated within a short time. Some observers may have thought that SCA is the same as a heart attack, but it is not. For such patients, an external cardiac defibrillator can deliver a shock through the heart to restore its normal rhythm. The problem is that it is hard for an external cardiac defibrillator to be brought to the patient within that short time. One solution, therefore, is for such patients to be given a WMS that implements a WCD. This solution is at least temporary and, after a while, such as two months, the patient may instead receive a surgically implantable cardioverter defibrillator (“ICD”), which would then become a permanent solution.

A WMS that implements a WCD typically includes a harness, vest, belt, or other garment that the patient is to wear. The WMS system further includes additional components that are coupled to the harness, vest, or other garment. Alternately, these additional components may be adhered to the patient's skin by adhesive. These additional components include a unit that has a defibrillator, and sensing and therapy electrodes. When the patient wears this WMS, the sensing electrodes may make good electrical contact with the patient's skin and therefore can help sense the patient's Electrocardiogram (“ECG”). If the unit detects a shockable heart arrhythmia from the ECG, then the unit delivers an appropriate electric shock to the patient's body through the therapy electrodes. The shock can pass through the patient's heart and may restore its normal rhythm, thus saving their life.

Generally, WCDs are designed with desire to minimize the size and weight of the system while still providing the required functionality. The energy storage capacitor is typically a relatively large component of the system that is selected to meet specifications such as, for example, energy storage capacity, voltage rating, etc. The stored charge is used to deliver one or more shocks to a patient. The energy storage capacitor is typically specified with a capacitance and a voltage rating sufficient to be charged to a specified voltage and deliver shocks of a specified minimum energy to a patient having a specified impedance.

All subject matter discussed in this Background section of this document is not necessarily prior art, and may not be presumed to be prior art simply because it is presented in this Background section. Additionally, any reference to any prior art in this description is not, and should not be taken as, an acknowledgement or any form of suggestion that such prior art forms parts of the common general knowledge in any art in any country. Along these lines, any recognition of problems in the prior art discussed in this Background section or associated with such subject matter should not be treated as prior art, unless expressly stated to be prior art. Rather, the discussion of any subject matter in this Background section should be treated as part of the approach taken towards the particular problem by the inventors. This approach in and of itself may also be inventive.

### SUMMARY

The present description gives instances of apparatus and methods for charging and specifying a capacitor for storing energy in a wearable cardioverter defibrillator. In one aspect, WCD circuitry is provided that includes a power source such as a battery coupled to a charger that provides charge energy to an energy storage capacitor having a rated voltage. Control circuitry is configured to implement a multi-stage charging scheme under which the energy storage capacitor is charged to a first intermediate voltage below its rated voltage during a first charging stage and charged to a second voltage above the rated voltage during a second charging stage. In response to detection of potential shockable rhythms the capacitor is charged to an intermediate voltage, while upon confirmation of shockable rhythms the capacitor may be charged to the second voltage, which is then used to deliver a shock to a patient.

These and other features and advantages of the claimed invention will become more readily apparent in view of the embodiments described and illustrated in this specification, namely in this written specification and the associated drawings.

### DESCRIPTION OF THE DRAWINGS

FIG. 1 is a diagram of sample components of a wearable medical system (“WMS”) that implements a wearable cardioverter defibrillator (“WCD”), and which is made according to embodiments.

FIG. 2A is a diagram showing a view of the inside of a sample garment embodiment that can be a support structure of a WMS that implements a WCD, such as that of FIG. 1.

FIG. 2B is a diagram showing a view of the outside of the sample garment of FIG. 2A.

FIG. 2C is a diagram showing a front view of how the sample garment of FIGS. 2A and 2B is intended to be worn by a patient.

FIG. 2D is a diagram showing a back view of how the sample garment of FIGS. 2A and 2B is intended to be worn by a patient.

FIG. 3 is a diagram showing a partial front view of another patient wearing a sample garment embodiment of an alternate style as worn by a patient, and which can be a support structure of a WMS that implements a WCD such as that of FIG. 1.

FIG. 4 is a diagram showing sample embodiments of electronic components of a WMS that implements a WCD, and which can be used with the garment of FIG. 2A or of FIG. 3.

FIG. 5 is a diagram showing sample components of a unit of FIG. 1, which is made according to embodiments.

FIG. 6 is a top-level functional block diagram of a system implementing a multi-stage charging scheme for charging an energy storage capacitor, according to one embodiment.

FIG. 7 is a flowchart illustrating an exemplary process to charge an energy storage capacitor using a multi-stage charging scheme and deliver shocks to an ambulatory patient wearing a WDC, according to one embodiment.

FIG. 8 is a diagram illustrating a process for specifying the energy storage capacitor, according to one embodiment.

FIG. 9A and FIG. 9B collectively illustrate a diagram of an extended confirmation time for ventricular tachycardia (VT) in accordance with one or more embodiments.

### DETAILED DESCRIPTION

Embodiments of apparatus and methods for charging and specifying a capacitor for storing charge in a WCD are described herein. In the following description, numerous specific details are set forth to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

For clarity, individual components in the Figures herein may also be referred to by their labels in the Figures, rather than by a particular reference number. Additionally, reference numbers referring to a particular type of component (as opposed to a particular component) may be shown with a reference number followed by “(typ)” meaning “typical.” It will be understood that the configuration of these components will be typical of similar components that may exist but are not shown in the drawing Figures for simplicity and clarity or otherwise similar components that are not labeled with separate reference numbers. Conversely, “(typ)” is not to be construed as meaning the component, element, etc. is typically used for its disclosed function, implement, purpose, etc.

A WMS that implements a wearable cardioverter defibrillator according to embodiments may protect an ambulatory patient by electrically restoring normal heart rhythm if needed. Such a WMS may have a number of components. These components can be provided separately as modules that can be interconnected, or can be combined with other components, and so on. Examples are now described.

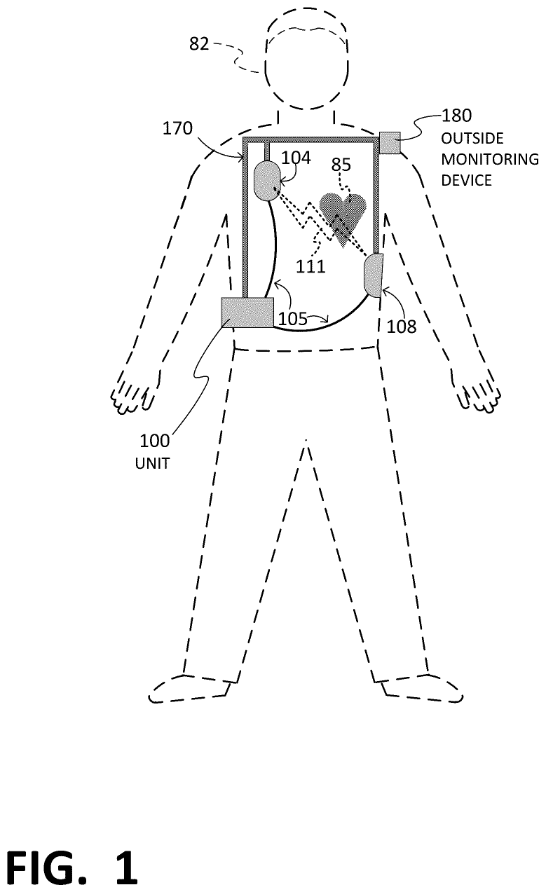

FIG. 1 depicts a patient 82. The patient 82 may also be referred to as the person 82 and/or wearer 82, since the patient 82 is wearing components of the WMS. The patient 82 is ambulatory, which means that, while wearing the wearable component(s) of the WMS, the patient 82 can walk around, be in a vehicle, and so on. In other words, the patient 82 is not necessarily bed-ridden. While the patient 82 may be considered to be also a “user” of the WMS, this definition is not exclusive to the patient 82. For instance, a user of the WMS may also be a clinician such as a doctor, nurse, emergency medical technician (EMT), or other similarly tasked and/or empowered individual or group of individuals. In some cases, a user may even be a bystander. The particular context of these and other related terms within this description should be interpreted accordingly.

A WMS that implements a WCD according to embodiments can be configured to defibrillate the patient who is wearing the designated components of the WMS. Defibrillating can be by the WMS delivering an electrical charge to the patient's body in the form of an electric shock. The electric shock can be delivered in one or more pulses.

In particular, FIG. 1 also depicts components of a WMS that implements a WCD and is made according to embodiments. One such component is a support structure 170 that is wearable by the ambulatory patient 82. Accordingly, the support structure 170 can be configured to be worn by the ambulatory patient 82 for at least several hours per day, and also during the night. That, for at least several days, and maybe even a few months. It will be understood that the support structure 170 is shown only generically in FIG. 1, and in fact partly conceptually. FIG. 1 is provided merely to illustrate concepts about the support structure 170, and is not to be construed as limiting how the support structure 170 is implemented, or how it is worn.

The support structure 170 can be implemented in many different ways. For example, it can be implemented in a single component or a combination of multiple components. In embodiments, the support structure 170 could include a vest, a half-vest, a garment, etc. In such embodiments such items can be worn similarly to analogous articles of clothing. In embodiments, the support structure 170 could include a harness, one or more belts or straps, etc. In such embodiments, such items can be worn by the patient around the torso, hips, over the shoulder, etc. In embodiments, the support structure 170 can include a container or housing, which can even be waterproof. In such embodiments, the support structure can be worn by being attached to the patient's body by adhesive material, for example as shown and described in U.S. Pat. No. 8,024,037. The support structure 170 can even be implemented as described for the support structure of US Pat. App. No. US2017/0056682, which is incorporated herein by reference. Of course, in such embodiments, the person skilled in the art will recognize that additional components of the WMS can be in the housing of a support structure instead of being attached externally to the support structure, for example as described in the US2017/0056682 document. There can be other examples.

The embodiments of FIG. 1 include a sample unit 100. In embodiments, the unit 100 is sometimes called a main electronics module. In embodiments, the unit 100 implements an external defibrillator. In embodiments, the unit 100 implements an external pacemaker instead of, or in addition to, an external defibrillator. In embodiments that include a pacemaker, the WMS may detect when the patient's heart rhythm slows down or when the patient has asystole, and the pacemaker may pace to increase the heart rate.

The embodiments of FIG. 1 also include sample therapy electrodes 104, 108, which are electrically coupled to unit 100 via electrode leads 105. The therapy electrodes 104, 108 are also called defibrillation electrodes or just electrodes. The therapy electrodes 104, 108 can be configured to be worn by the patient 82 in a number of ways. For instance, the unit 100 and the therapy electrodes 104, 108 can be coupled to the support structure 170, directly or indirectly. In other words, the support structure 170 can be configured to be worn by the ambulatory patient 82 so as to maintain at least one of the therapy electrodes 104, 108 on the body of the ambulatory patient 82, while the patient 82 is moving around, etc. The therapy electrodes 104, 108 can be thus maintained on the body by being attached to the skin of the patient 82, simply pressed against the skin directly or through garments, etc. In some embodiments the therapy electrodes 104, 108 are not necessarily pressed against the skin, but become biased that way upon sensing a condition that could merit intervention by the WMS. In addition, many of the components of the unit 100 can be considered coupled to the support structure 170 directly, or indirectly, via at least one of the therapy electrodes 104, 108.

When the therapy electrodes 104, 108 make good electrical contact with the body of the patient 82, the unit 100 can administer, via the therapy electrodes 104, 108, a brief, strong electric pulse 111 through the body. The pulse 111 is also known as defibrillation pulse, shock, defibrillation shock, therapy, electrotherapy, therapy shock, etc. The pulse 111 is intended to go through and restore normal sinus rhythm to the heart 85, in an effort to save the life of the patient 82. The defibrillation pulse 111 can have an energy suitable for its purpose, such as at least 100 Joule (“J”), 200J, 300J, and so on. For pacemaker embodiments, the pulse 111 could alternately be depicting a pacing pulse. At least some of the stored electrical charge can be caused to be discharged via at least two of the therapy electrodes 104, 108 through the ambulatory patient 82, so as to deliver to the ambulatory patient 82 a sequence of pacing pulses. The pacing pulses may be periodic, and thus define a pacing period and the pacing rate. There is no requirement, however, that the pacing pulses be exactly periodic. A pacing pulse can have an energy suitable for its purpose, such as at most 10J, 5J, usually about 2J, and so on. The pacemaker therefore is delivering current to the heart to start a heartbeat. In either case, the pulse 111 has a waveform suitable for this purpose.

A prior art defibrillator typically decides whether to defibrillate or not based on an ECG signal of the patient. However, the unit 100 may initiate defibrillation, or hold-off defibrillation, based on a variety of inputs, with the ECG signal merely being one of these inputs.

A WMS that implements a WCD according to embodiments can collect data about one or more parameters of the patient 82. For collecting such data, the WMS may optionally include at least an outside monitoring device 180. The device 180 is called an “outside” device because it could be provided as a standalone device, for example not within the housing of the unit 100. The device 180 can be configured to sense or monitor at least one local parameter. A local parameter can be a parameter of the patient 82, or a parameter of the WMS, or a parameter of the environment, as described later in this document.

For some of these parameters, the device 180 may include one or more sensors or transducers. Each one of such sensors can be configured to sense a parameter of the patient 82, or of the environment, and to render an input responsive to the sensed parameter. In some embodiments the input is quantitative, such as values of a sensed parameter: in other embodiments the input is qualitative, such as informing whether or not a threshold is crossed, and so on. Such inputs about the patient 82 are also called physiological inputs and patient inputs. In embodiments, a sensor can be construed more broadly, as encompassing more than one individual sensors.

Optionally, the device 180 is physically coupled to the support structure 170. In addition, the device 180 may be communicatively coupled with other components that are coupled to the support structure 170, such as with the unit 100. Such communication can be implemented by the device 180 itself having a communication module, as will be deemed applicable by a person skilled in the art in view of this description.

A WMS that implements a WCD according to embodiments preferably includes sensing electrodes, which can sense an ECG of the patient. In embodiments, the device 180 stands for such sensing electrodes. In those embodiments, the sensed parameter of the patient 82 is the ECG of the patient, the rendered input can be time values of a waveform of the ECG signal, and so on.

In embodiments, one or more of the components of the shown WMS may be customized for the patient 82. This customization may include a number of aspects. For instance, the support structure 170 can be fitted to the body of the patient 82. For another instance, baseline physiological parameters of the patient 82 can be measured for various scenarios, such as when the patient is lying down (various orientations), sitting, standing, walking, running, and so on. These baseline physiological parameters can be the heart rate of the patient 82, motion detector outputs, one for each scenario, etc. The measured values of such baseline physiological parameters can be used to customize the WMS, in order to make its diagnoses more accurate, since patients' bodies differ from one another. Of course, such parameter values can be stored in a memory of the WMS, and so on. Moreover, a programming interface can be made according to embodiments, which receives such measured values of baseline physiological parameters.

The support structure 170 is configured to be worn by the ambulatory patient 82 so as to maintain the therapy electrodes 104, 108 on a body of the patient 82. As mentioned before, the support structure 170 can be advantageously implemented by clothing or one or more garments. Such clothing or garments do not have the function of covering a person's body as a regular clothing or garments do, but the terms “clothing” and “garment” are used in this art for certain components of the WMS intended to be worn on the human body in the same way as clothing and garments are. In fact, such clothing and garments of a WMS can be of different sizes for different patients, and even be custom-fitted around the human body. And, regular clothing can often be worn over portions or all of the support structure 170. Examples of the support structure 170 are now described.

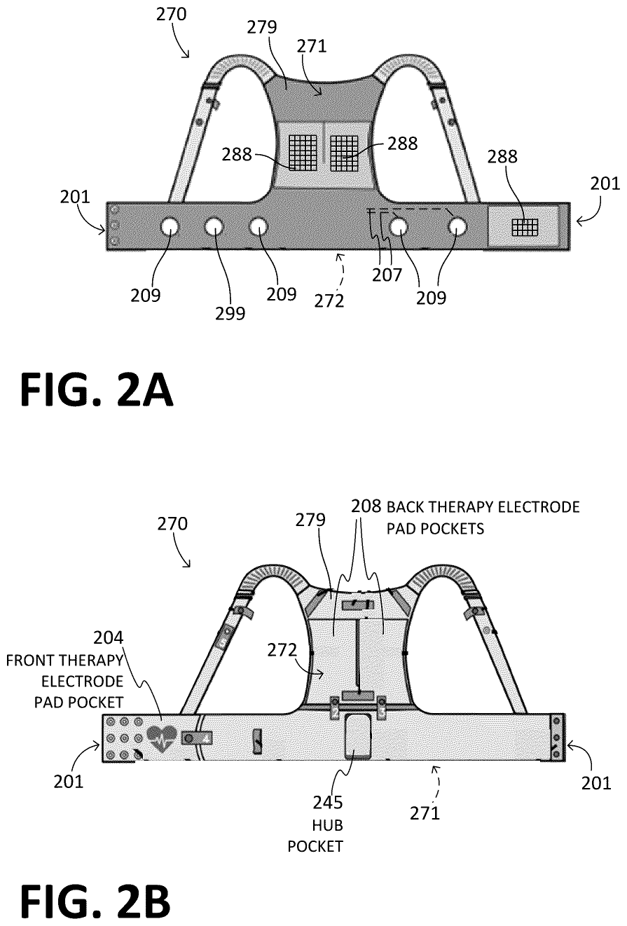

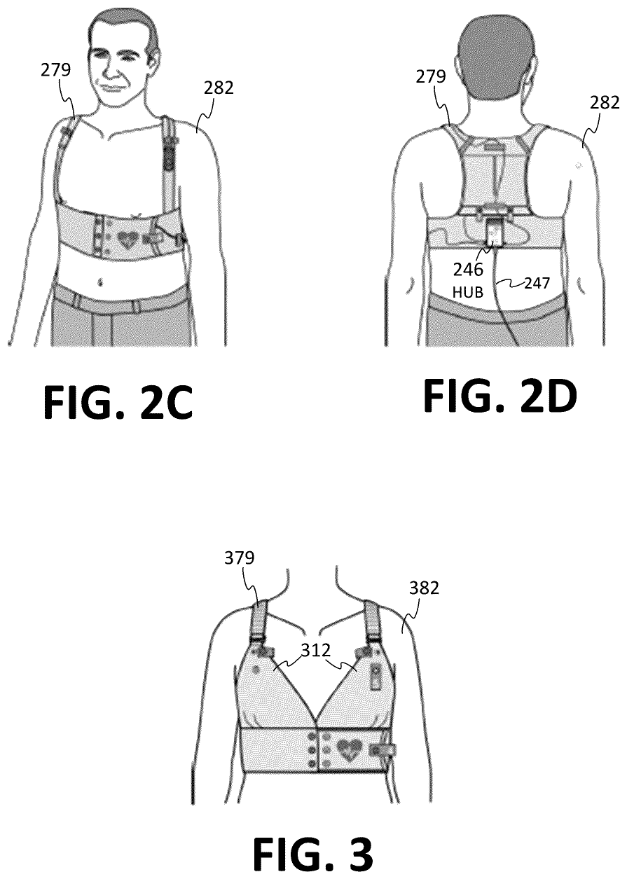

FIG. 2A shows a support structure 270 of a WMS that implements a WCD, such as the support structure 170 of FIG. 1. The support structure 270 is implemented by a vest-like wearable garment 279 that is shown flat, as if placed on a table. The inside side 271 of the garment 279 is seen as one looks at the diagram from the top, and it is the side contacting the body of the wearer when the garment 279 is worn. The outside side 272 of the garment 279 is opposite the inside side 271. To be worn, tips 201 can be brought together while surrounding the torso, and affixed to each other, either at their edges or partly overlapping. Appropriate mechanisms can hold together the tips 201, such as buttons/snaps, hooks and loops, Velcro® material, and so on.

The garment 279 can be made of suitable combinations of materials, such as fabric, linen, plastic, and so on. In places, the garment 279 can have two adjacent surfaces for defining between them pockets for the pads of the electrodes, for enclosing the leads or wires of the electrodes, and so on. Moreover, in FIG. 2A one can see meshes 288 which are the interior side of pockets accessible from the outside. The meshes can be made from flexible material such as loose netting, and so on.

ECG signals in a WMS that implements a WCD may sometimes include too much electrical noise for analyzing the ECG signal. To ameliorate the problem, multiple ECG sensing electrodes are provided in embodiments. These multiple ECG sensing electrodes define different vectors for sensing ECG signals along different ECG channels. These different ECG channels therefore present alternative options for analyzing the patient's ECG signal. The patient impedance along each ECG channel may also be sensed, and thus be part of the patient input.

In the example of FIG. 2A, multiple ECG sensing electrodes 209 are provided, which can be seen protruding from the inside surface of the garment 279. These ECG sensing electrodes 209 can be affixed to the inside surface of the garment 279, while their leads or wires 207 can be located mostly or completely within the garment 279. These ECG sensing electrodes 209 are intended to contact the skin of the person when the garment 279 is worn, and can be made from suitable material for good electrical contact. Such a material can be a metal, such as silver, or other sufficiently conductive materials. An additional ECG-sensing electrode 299 may play the role of a Right Leg Drive (“RLD”) in the ECG analysis. In this context “RLD” is a custom electrical term, and embodiments do not require that the electrode 299 be actually placed on a leg of the patient.

FIG. 2B shows the outside side 272 of the garment 279. One can appreciate that pockets are included that are accessible from the outside, such as a hub pocket or receptacle 245. In addition, a pocket 204 is provided for a front therapy electrode pad, plus two pockets 208 are provided for two back therapy electrode pads. The pads of the therapy electrodes can be placed in the pockets 204, 208, and contact the skin of the patient through the respective meshes 288 that were seen in FIG. 2A. The electrical contact can be facilitated by conductive fluid that can be deployed in the area, when the time comes for a shock.

FIG. 2C is a diagram showing a front view of how the garment 279 would be worn by a patient 282. It will be appreciated that the previously described ECG sensing electrodes 209, 299 of FIG. 2A are maintained against the body of the patient 282 from the inside side of the garment 279, and thus are not visible in FIG. 2C.

FIG. 2D is a diagram showing the back view of FIG. 2C. A hub 246 has been placed in the hub pocket 245 that is shown in FIG. 2B. A cable 247 emerges from the hub 246, which can be coupled with a unit for the system, as described later in this document.

FIGS. 2A-2D do not show any physical support for a unit such as the unit 100 of FIG. 1. In these embodiments, such a unit may be carried in a purse, on a belt, by a strap over the shoulder, or additionally by further adapting the garment 279, and so on.

FIG. 3 is a diagram showing a partial front view of another patient 382 wearing another garment 379. The garment 379 is of an alternate style than the garment 279, in that it further includes breast support receptacles 312, as was described for instance in U.S. Pat. No. 10,926,080. This style of garment may be more comfortable if the patient 382 is a woman.

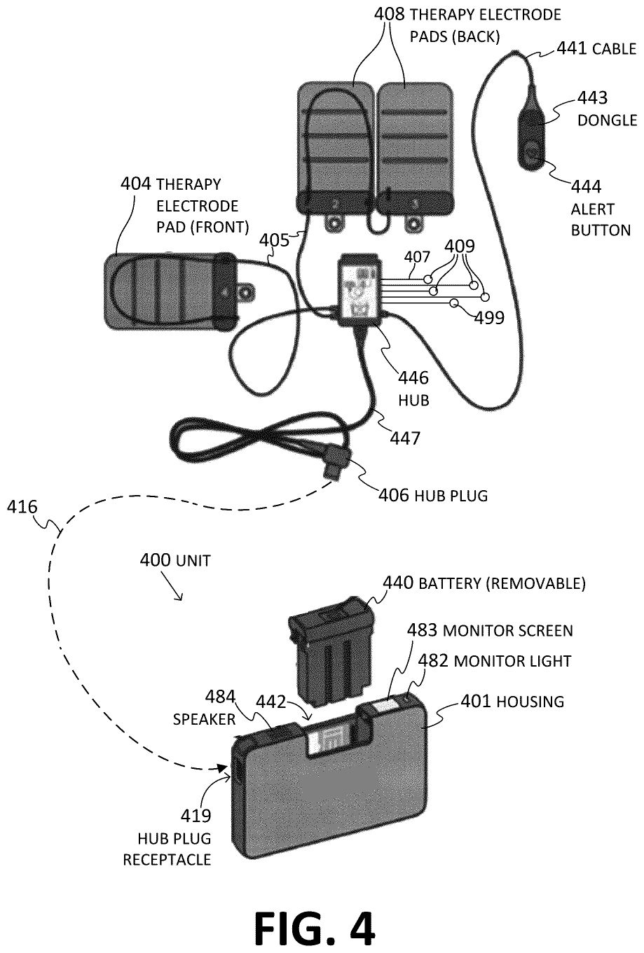

FIG. 4 shows sample electronic components that can be used with the garments 279, 379. The components of FIG. 4 include a unit 400, shown at the lower portion of FIG. 4. The unit 400 includes a housing 401, and a hub plug receptacle 419 at the housing 401.

The unit 400 includes a battery opening 442 at the housing 401. The battery opening 442 is configured to receive a removable battery 440. A system according to embodiments can have two identical such batteries 440, one plugged into the housing 401 while another one (not shown) is being charged by a charger (not shown). The batteries can then be interchanged when needed.

The unit 400 also includes devices for implementing a user interface. In this example, these devices include a monitor light 482, a monitor screen 483 and a speaker 484. Additional devices may include a vibrating mechanism, and so on.

The unit 400 can implement many of the functions of the unit 100 of FIG. 1. In the embodiment of FIG. 4, however, some of the functions of the unit 100 are implemented instead by a separate hub 446, which can be connected to the unit 400. The hub 446 is smaller and lighter than the unit 400, and can accommodate multiple electrical connections to other components of FIG. 4. A cable 447, similar to the cable 247 of FIG. 2D, emerges from the hub 446 and terminates in a hub plug 406. The hub plug 406 can be plugged into the hub plug receptacle 419 of the unit 400 according to an arrow 416.

ECG sensing electrodes 409, 499, plus their wires or leads 407 are further shown conceptually in FIG. 4 for completeness. The wires or leads 407 that can be configured to be coupled to the hub 446.

The components of FIG. 4 also include the therapy electrode pads 404, 408. The therapy electrode pad 404 can be inserted into the pocket 204 of FIG. 2B, while the therapy electrode pads 408 can be inserted into the pockets 208 of FIG. 2B. The shock is generated between the therapy electrode pad 404 and the therapy electrode pads 408 taken together. Indeed, the therapy electrode pads 408 are electrically connected to each other. The therapy electrode pads 404, 408, have leads 405, which can be configured to be coupled to the hub 446.

The components of FIG. 4 further include a dongle 443 with an alert button 444. The dongle 443 can be configured to be coupled to the hub 446 via a cable 441. The alert button 444 can be used by the patient to give emergency input to the WMS. For instance, the alert button 444 can be used by the patient to notify the system that the patient is actually alive and an imminent shock is not actually needed, which may otherwise happen in the event of a false positive detection of a shockable heart rhythm of the patient.

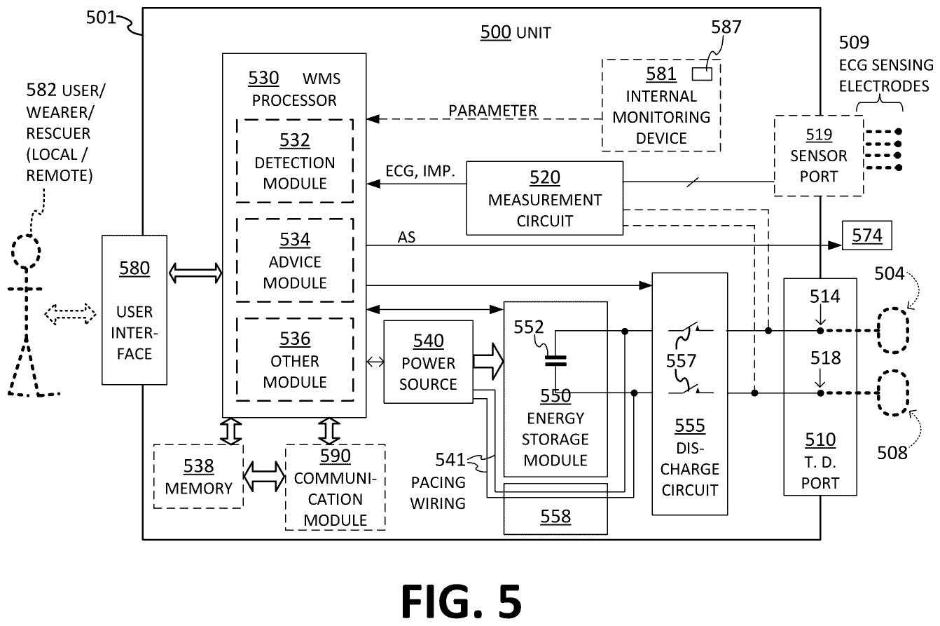

FIG. 5 shows a sample unit 500, which could be the unit 100 of FIG. 1. The unit 500 implements an external defibrillator and/or a pacemaker. The sample unit 500 thus combines the functions of the unit 400 and of the hub 446 of FIG. 4. The components shown in FIG. 5 can be provided in a housing 501, which may also be referred to as casing 501.

The unit 500 may include a user interface (UI) 580 for a user 582. User 582 can be the patient 82, also known as patient 582, also known as the wearer 582. Or, the user 582 can be a local rescuer at the scene, such as a bystander who might offer assistance, or a trained person. Or, the user 582 might be a remotely located trained caregiver in communication with the WMS, such as a clinician.

The user interface 580 can be made in a number of ways. The user interface 580 may include output devices, which can be visual, audible or tactile, for communicating to a user by outputting images, sounds or vibrations. Images, sounds, vibrations, and anything that can be perceived by user 582 can also be called human-perceptible indications. As such, an output device according to embodiments can be configured to output a human-perceptible indication (HPI). Such HPIs can be used to alert the patient, sound alarms that may be intended also for bystanders, and so on. There are many instances of output devices. For example, an output device can be a light that can be turned on and off, a screen to display what is sensed, detected and/or measured, and provide visual feedback to the local rescuer 582 for their resuscitation attempts, and so on. Another output device can be a speaker, which can be configured to issue voice prompts, alerts, beeps, loud alarm sounds and/or words, and so on. These can also be for bystanders, when defibrillating or just pacing, and so on. Examples of output devices are the monitor light 482, the monitor screen 483 and the speaker 484 of the unit 400 seen in FIG. 4.

The user interface 580 may further include input devices for receiving inputs from users. Such users can be the patient 82, 582, perhaps a local trained caregiver or a bystander, and so on. Such input devices may include various controls, such as pushbuttons, keyboards, touchscreens, one or more microphones, and so on. An input device can be a cancel switch, which is sometimes called an “I am alive” switch or “live man” switch or divert switch. In some embodiments, actuating the cancel switch can prevent the impending delivery of a shock, or of pacing pulses. In particular, in some embodiments a speaker of the WMS is configured to output a warning prompt prior to an impending or planned defibrillation shock or a sequence of pacing pulses being caused to be delivered, and the cancel switch is configured to be actuated by the ambulatory patient 82 in response to the warning prompt being output. In such embodiments, the impending or planned defibrillation shock or sequence of the pacing pulses is not caused to be delivered. An example of a cancel switch was the alert button 444 seen in FIG. 4.

The unit 500 may include an internal monitoring device 581. The device 581 is called an “internal” device because it is incorporated within the housing 501. The monitoring device 581 can sense or monitor patient parameters such as patient physiological parameters, system parameters and/or environmental parameters, all of which can be called patient data. In other words, the internal monitoring device 581 can be complementary of, or an alternative to, the outside monitoring device 180 of FIG. 1. Allocating which of the parameters are to be monitored by which of the monitoring devices 180, 581 can be done according to design considerations. The device 581 may include one or more sensors, as also described elsewhere in this document.

Patient parameters may include patient physiological parameters. Patient physiological parameters may include, for example and without limitation, those physiological parameters that can be of any help in detecting by the WMS whether or not the patient is in need of a shock or other intervention or assistance. Patient physiological parameters may also optionally include the patient's medical history, event history and so on. Examples of such parameters include the above-described electrodes to detect the ECG, blood oxygen level, blood flow, blood pressure, blood perfusion, pulsatile change in light transmission or reflection properties of perfused tissue, heart sounds, heart wall motion, breathing sounds and pulse. Accordingly, the monitoring devices 180, 581 may include one or more sensors or transducers configured to acquire patient physiological signals. Examples of such sensors and transducers include one or more electrodes to detect ECG data, a perfusion sensor, a pulse oximeter, a device for detecting blood flow (e.g. a Doppler device), a sensor for detecting blood pressure (e.g. a cuff), an optical sensor, illumination detectors and sensors perhaps working together with light sources for detecting color change in tissue, a motion sensor, a device that can detect heart wall movement, a sound sensor, a device with a microphone, an SpO2 sensor, and so on. In view of this disclosure, it will be appreciated that such sensors can help detect the patient's pulse, and can therefore also be called pulse detection sensors, pulse sensors, and pulse rate sensors. In addition, a person skilled in the art may implement other ways of performing pulse detection.

In some embodiments, the local parameter reflects a trend that can be detected in a monitored physiological parameter of the patient 82, 582. Such a trend can be detected by comparing values of parameters at different times over short and long terms. Parameters whose detected trends can particularly help a cardiac rehabilitation program include: a) cardiac function (e.g. ejection fraction, stroke volume, cardiac output, etc.); b) heart rate variability at rest or during exercise; c) heart rate profile during exercise and measurement of activity vigor, such as from the profile of an accelerometer signal and informed from adaptive rate pacemaker technology; d) heart rate trending; e) perfusion, such as from SpO2, CO2, or other parameters such as those mentioned above, f) respiratory function, respiratory rate, etc.; g) motion, level of activity; and so on. Once a trend is detected, it can be stored and/or reported via a communication link, along perhaps with a warning if warranted. From the report, a physician monitoring the progress of the patient 82, 582 will know about a condition that is either not improving or deteriorating.

Patient state parameters include recorded aspects of the patient 582, such as motion, posture, whether they have spoken recently plus maybe also what they said, and so on, plus optionally the history of these parameters. Or, one of these monitoring devices could include a location sensor such as a Global Positioning System (GPS) location sensor. Such a sensor can detect the location, plus a speed of the patient can be detected as a rate of change of location over time. Many motion detectors output a motion signal that is indicative of the motion of the detector, and thus of the patient's body. Patient state parameters can be very helpful in narrowing down the determination of whether SCA is indeed taking place.

A WMS made according to embodiments may thus include a motion detector. In embodiments, a motion detector can be implemented within the outside monitoring device 180 or within the internal monitoring device 581. A motion detector of a WMS according to embodiments can be configured to detect a motion event. A motion event can be defined as is convenient, for example a change in posture or motion from a baseline posture or motion, etc. In such cases, a sensed patient parameter is motion. Such a motion detector can be made in many ways as is known in the art, for example by using an accelerometer and so on. In this example, a motion detector 587 is implemented within the monitoring device 581.

System parameters of a WMS can include system identification, battery status, system date and time, reports of self-testing, records of data entered, records of episodes and intervention, and so on. In response to the detected motion event, the motion detector may render or generate, from the detected motion event or motion, a motion detection input that can be received by a subsequent device or functionality.

Environmental parameters can include ambient temperature and pressure. Moreover, a humidity sensor may provide information as to whether or not it is likely raining. Presumed patient location could also be considered an environmental parameter. The patient location could be presumed, if the monitoring device 180 or 581 includes a GPS location sensor as per the above, and if it is presumed or sensed that the patient is wearing the WMS.

The unit 500 includes a therapy delivery port 510 and a sensor port 519 in the housing 501. In contrast, in FIG. 4 these ports are located at the hub 446.

In FIG. 5, the therapy delivery port 510 can be a socket in the housing 501, or other equivalent structure. The therapy delivery port 510 includes electrical nodes 514, 518. Therapy electrodes 504, 508 are shown, which can be as the therapy electrodes 104, 108. Leads of the therapy electrodes 504, 508, such as the leads 105 of FIG. 1, can be plugged into the therapy delivery port 510, so as to make electrical contact with the nodes 514, 518, respectively. It is also possible that the therapy electrodes 504, 508 are connected continuously to the therapy delivery port 510, instead. Either way, the therapy delivery port 510 can be used for guiding, via electrodes, to the wearer at least some of the electrical charge that has been stored in an energy storage module 550 that is described more fully later in this document. When thus guided, the electric charge will cause the shock 111 to be delivered.

The sensor port 519 is also in the housing 501, and is also sometimes known as an ECG port. The sensor port 519 can be adapted for plugging in the leads of ECG sensing electrodes 509. The ECG sensing electrodes 509 can be as the ECG sensing electrodes 209. The ECG sensing electrodes 509 in this example are distinct from the therapy electrodes 504, 508. It is also possible that the sensing electrodes 509 can be connected continuously to the sensor port 519, instead. The electrodes 509 can be types of transducers that can help sense an ECG signal of the patient, e.g., a 12-lead signal, or a signal from a different number of leads, especially if they make good electrical contact with the body of the patient and in particular with the skin of the patient. As with the therapy electrodes 504, 508, the support structure can be configured to be worn by the patient 582 so as to maintain the sensing electrodes 509 on a body of the patient 582. For example, the sensing electrodes 509 can be attached to the inside of the support structure 170 for making good electrical contact with the patient, similarly with the therapy electrodes 504, 508.

Optionally a WMS according to embodiments also includes a fluid that it can deploy automatically between the electrodes and the patient's skin. The fluid can be conductive, such as by including an electrolyte, for establishing a better electrical contact between the electrodes and the skin. Electrically speaking, when the fluid is deployed, the electrical impedance between each electrode and the skin is reduced. Mechanically speaking, the fluid may be in the form of a low-viscosity gel. As such, it will not flow too far away from the location it is released. The fluid can be used for both the therapy electrodes 504, 508, and for the sensing electrodes 509.

The fluid may be initially stored in a fluid reservoir, not shown in FIG. 5. Such a fluid reservoir can be coupled to the support structure. In addition, a WMS according to embodiments further includes a fluid deploying mechanism 574. The fluid deploying mechanism 574 can be configured to cause at least some of the fluid to be released from the reservoir, and be deployed near one or both of the patient body locations to which the therapy electrodes 504, 508 are configured to be attached to the patient's body. In some embodiments, the fluid deploying mechanism 574 is activated prior to the electrical discharge responsive to receiving an activation signal AS from the processor 530, which is described more fully later in this document.

In some embodiments, unit 500 also includes a measurement circuit 520, as one or more of its modules working together with its sensors and/or transducers. The measurement circuit 520 senses one or more electrical physiological signals of the patient from sensor port 519, if provided. Even if the unit 500 lacks a sensor port, the measurement circuit 520 may optionally obtain physiological signals through the nodes 514, 518 instead, when the therapy electrodes 504, 508 are attached to the patient. In these cases, the input reflects an ECG measurement. The patient parameter can be an ECG, which can be sensed as a voltage difference between electrodes 504, 508. In addition, the patient parameter can be an impedance (IMP. or Z), which can be sensed between the electrodes 504, 508 and/or between the connections of the sensor port 519 considered pairwise as channels. Sensing the impedance can be useful for detecting, among other things, whether these electrodes 504, 508 and/or the sensing electrodes 509 are not making good electrical contact with the patient's body at the time. These patient physiological signals may be sensed when available. The measurement circuit 520 can then render or generate information about them as inputs, data, other signals, etc. As such, the measurement circuit 520 can be configured to render a patient input responsive to a patient parameter sensed by a sensor. In some embodiments, the measurement circuit 520 can be configured to render a patient input, such as values of an ECG signal, responsive to the ECG signal sensed by the ECG sensing electrodes 509. More strictly speaking, the information rendered by the measurement circuit 520 is output from it, but this information can be called an input because it is received as an input by a subsequent stage, device or functionality.

Unit 500 also includes a processor 530. The processor 530 may be implemented in a number of ways. Such ways include, by way of example and not of limitation, digital and/or analog processors such as microprocessors and Digital Signal Processors (DSPs), controllers such as microcontrollers, software running in a machine, programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

The processor 530 may include, or have access to, a non-transitory storage medium, such as a memory 538 that is described more fully later in this document. Such a memory can have a non-volatile component for storage of machine-readable and machine-executable instructions. A set of such instructions can also be called a program. The instructions, which may also be referred to as “software,” generally provide functionality by performing acts, operations and/or methods as may be disclosed herein or understood by one skilled in the art in view of the disclosed embodiments. In some embodiments, and as a matter of convention used herein, instances of the software may be referred to as a “module” and by other similar terms. Generally, a module includes a set of the instructions so as to offer or fulfill a particular functionality. Embodiments of modules and the functionality delivered are not limited by the embodiments described in this document.

The processor 530 can be considered to have a number of modules. One such module can be a detection module 532. The detection module 532 can include a Ventricular Fibrillation (VF) detector. The patient's sensed ECG from measurement circuit 520, which can be available as inputs, data that reflect values, or values of other signals, may be used by the VF detector to determine whether the patient is experiencing VF. Detecting VF is useful, because VF typically results in SCA. The detection module 532 can also include a Ventricular Tachycardia (VT) detector for detecting VT, and so on.

Another such module in processor 530 can be an advice module 534, which generates advice for what to do. The advice can be based on outputs of the detection module 532. There can be many types of advice according to embodiments. In some embodiments, the advice is a shock/no shock determination that processor 530 can make, for example via advice module 534. The shock/no shock determination can be made by executing a stored Shock Advisory Algorithm. A Shock Advisory Algorithm can make a shock/no shock determination from one or more ECG signals that are sensed according to embodiments, and determine whether or not a shock criterion is met. The determination can be made from a rhythm analysis of the sensed ECG signal or otherwise. For example, there can be shock decisions for VF, VT, etc.

In perfect conditions, a very reliable shock/no shock determination can be made from a segment of the sensed ECG signal of the patient. In practice, however, the ECG signal is often corrupted by electrical noise, which makes it difficult to analyze. Too much noise sometimes causes an incorrect detection of a heart arrhythmia, resulting in a false alarm to the patient. Noisy ECG signals may be handled as described in U.S. Pat. No. 10,918,879 (“Wearable cardioverter defibrillator (WCD) system reacting to high-amplitude ECG noise”) and U.S. Pat. No. 11,103,717 (“Wearable cardioverter defibrillator (WCD) system reacting to high-frequency ECG noise”), which are incorporated herein by reference for all purposes.

The processor 530 can include additional modules, such as other module 536, for other functions. In addition, if the internal monitoring device 581 is indeed provided, the processor 530 may receive its inputs, etc.

The unit 500 optionally further includes a memory 538, which can work together with the processor 530. The memory 538 may be implemented in a number of ways. Such ways include, by way of example and not of limitation, volatile memories, Nonvolatile Memories (NVM), Read-Only Memories (ROM), Random Access Memories (RAM), magnetic disk storage media, optical storage media, smart cards, flash memory devices, any combination of these, and so on. The memory 538 is thus a non-transitory storage medium. The memory 538, if provided, can include programs for the processor 530, which the processor 530 may be able to read and execute. More particularly, the programs can include sets of instructions in the form of code, which the processor 530 may be able to execute upon reading. Executing is performed by physical manipulations of physical quantities, and may result in functions, operations, processes, acts, actions and/or methods to be performed, and/or the processor 530 to cause other devices or components or blocks to perform such functions, operations, processes, acts, actions and/or methods. The programs can be operational for the inherent needs of the processor 530, and can also include protocols and ways that decisions can be made by the advice module 534. In addition, the memory 538 can store prompts for the user 582, if this user is a local rescuer. Moreover, the memory 538 can store data. This data can include patient data, system data and environmental data, for example as learned by the internal monitoring device 581 and the outside monitoring device 180. The data can be stored in the memory 538 before it is transmitted out of the unit 500, or be stored there after it is received by the unit 500.

The unit 500 can optionally include a communication module 590, for establishing one or more wired or wireless communication links with other devices of other entities, such as a remote assistance center, Emergency Medical Services (EMS), and so on. The communication links can be used to transfer data and commands. The data may be patient data, event information, therapy attempted, CPR performance, system data, environmental data, and so on. For example, the communication module 590 may transmit wirelessly, e.g. on a daily basis, heart rate, respiratory rate, and other vital signs data to a server accessible over the internet, for instance as described in US App. Pub. No. 20140043149. This data can be analyzed directly by the patient's physician and can also be analyzed automatically by algorithms designed to detect a developing illness and then notify medical personnel via text, email, phone, etc. The module 590 may also include such interconnected sub-components as may be deemed necessary by a person skilled in the art, for example an antenna, portions of a processor, supporting electronics, outlet for a telephone or a network cable, etc.

The unit 500 may also include a power source 540, which is configured to provide electrical charge in the form of a current. To enable portability of the unit 500, the power source 540 typically includes a battery. Such a battery is typically implemented as a battery pack, which can be rechargeable or not. Sometimes a combination is used of rechargeable and non-rechargeable battery packs. An example of a rechargeable battery 540 was a battery 440 of FIG. 4. Other embodiments of the power source 540 can include an AC power override, for where AC power will be available, an energy-storing capacitor, and so on. Appropriate components may be included to provide for charging or replacing the power source 540. In some embodiments, the power source 540 is controlled and/or monitored by the processor 530.

The unit 500 may additionally include an energy storage module 550. The energy storage module 550 can be coupled to receive the electrical charge provided by the power source 540. The energy storage module 550 can be configured to store the electrical charge received by the power source 540. As such, the energy storage module 550 is where some electrical energy can be stored temporarily in the form of an electrical charge, when preparing it for discharge to administer a shock. In embodiments, the module 550 can be charged from the power source 540 to the desired amount of energy, for instance as controlled by the processor 530. In typical implementations, the module 550 includes a capacitor 552, which can be a single capacitor or a system of capacitors, and so on. In some embodiments, the energy storage module 550 includes a device that exhibits high power density. As described above, the capacitor 552 can store the energy in the form of an electrical charge, for delivering to the patient. In some embodiments, the energy storage module 550 may include a dump circuit or other circuitry (not shown) that may be selectively operated to dump some of the energy stored in capacitor 552.

As mentioned above, the patient is typically shocked when the shock criterion is met. In particular, in some embodiments the processor 530 is configured to determine from the patient input whether or not a shock criterion is met, and cause, responsive to the shock criterion being met, at least some of the electrical charge stored in the module 550 to be discharged via the therapy electrodes 104, 108 through the ambulatory patient 82 while the support structure is worn by the ambulatory patient 82 so as to deliver the shock 111 to the ambulatory patient 82. Delivering the electrical charge is also known as discharging and shocking the patient.

For causing the discharge, the unit 500 moreover includes a discharge circuit 555. When the decision is to shock, the processor 530 can be configured to control the discharge circuit 555 to discharge through the patient at least some of or all of the electrical charge stored in the energy storage module 550, especially in a desired waveform. When the decision is to merely pace, i.e., to deliver pacing pulses, the processor 530 can be configured to control the discharge circuit 555 to discharge through the patient at least some of the electrical charge provided by the power source 540. Since pacing requires lesser charge and/or energy than a defibrillation shock, in some embodiments pacing wiring 541 is provided from the power source 540 to the discharge circuit 555. The pacing wiring 541 is shown as two wires that bypass the energy storage module 550, and only go through a current-supplying circuit 558. As such, the energy for the pacing is provided by the power source 540 either via the pacing wiring 541, or through the energy storage module 550. And, in some embodiments where only a pacemaker is provided, the energy storage module 550 may not be needed if enough pacing current can be provided from the power source 540. Either way, discharging can be to the nodes 514, 518, and from there to the therapy electrodes 504, 508, so as to cause a shock to be delivered to the patient. The circuit 555 can include one or more switches 557. The switches 557 can be made in a number of ways, such as by an H-bridge, and so on. In some embodiments, different ones of the switches 557 may be used for a discharge where a defibrillation shock is caused to be delivered, than for a discharge where the much weaker pacing pulses are caused to be delivered. The circuit 555 could also be thus controlled via the processor 530, and/or the user interface 580.

The pacing capability can be implemented in a number of ways. ECG sensing may be done in the processor, as mentioned elsewhere in this document, or separately, for demand or synchronous pacing. In some embodiments, however, pacing can be asynchronous. Pacing can be software controlled, e.g., by managing the defibrillation path, or a separate pacing therapy circuit (not shown) could be included, which can receive the ECG sensing, via the circuit 520 or otherwise.

A time waveform of the discharge may be controlled by thus controlling discharge circuit 555. The amount of energy of the discharge can be controlled by how much energy storage module has been charged, and also by how long the discharge circuit 555 is controlled to remain open.

The unit 500 can optionally include other components.

As discussed above, the energy storage capacitor is typically specified with a capacitance and a voltage rating sufficient to be charged to a specified voltage and deliver shocks of a specified minimum energy to a patient having a specified impedance. Typical high voltage capacitors come with a voltage rating that indicates how much voltage can be applied to the capacitor continuously for an indefinite period while maintaining a specified reliability. Some types of capacitors can withstand much higher voltage than their continuous voltage rating if it is only applied for a limited amount of time. The shorter the amount of time, the higher the voltage can go.

In accordance with aspects of embodiments disclosed herein, apparatus and associated multi-stage charging methods are provided that enable WCDs to employ energy storage capacitors that have a lower voltage rating than used in existing WCDs, thereby reducing the size of the capacitors while also reducing the weight and/or bulk of the WCDs. Generally, WCDs do not require the energy storage capacitor to be continuously charged as long as it can be charged fully within a specified amount of time in-order to not delay therapy to the patient. Under one aspect, a WCD contains a Rhythm Recognition Detector (RRD) that monitors the heart rhythm to determine when shock therapy is required. The process the RRD uses to do this has different phases such as initial detection of a fast heart rate, a confirmation period where the algorithm evaluates the rate and waveform of the rhythm and a warning period to give the patient time to divert the shock if they are conscious. For example, in some embodiments such confirmation and warning periods are similar to those disclosed in U.S. Pat. No. 11,364,388, entitled “WCD System Operable to not Alarm When Detected Cardiac Arrhythmias are not Validated,” which is incorporated herein by reference for all purposes. In accordance with embodiments of the present disclosure, these periods of time can be used to charge the capacitor in stages to both ensure that it gets to full voltage before the shock needs to be delivered and also minimize the amount of time that it is held at full voltage.

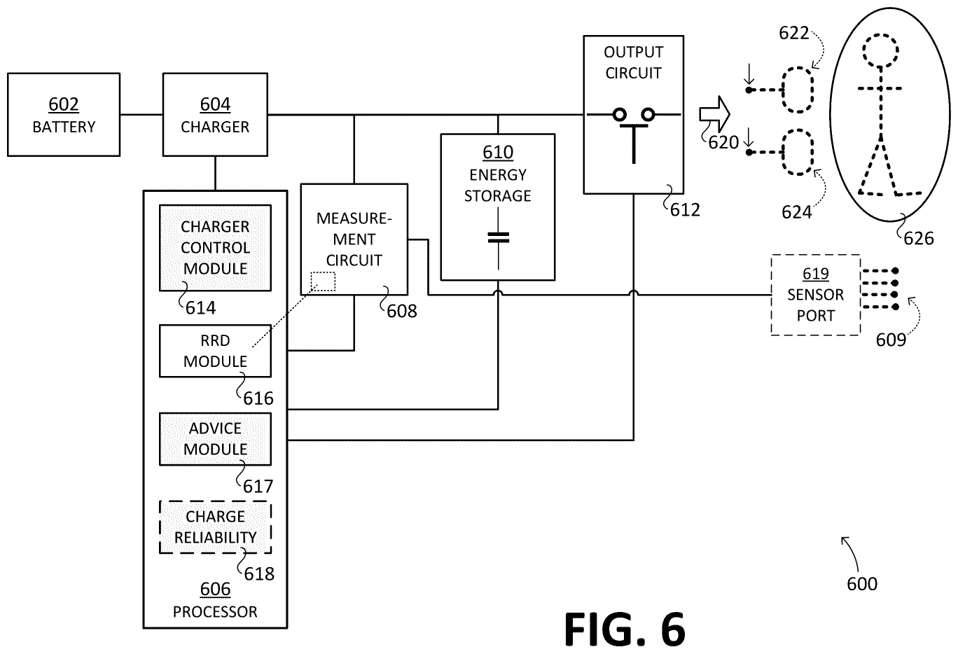

FIG. 6 shows a top-level functional block diagram of a system 600 illustrating selective components of WCD configured to support multi-stage charging, according to one embodiment. System 600 includes a battery 602 that provides power to a charger 604, which is controlled by a processor 606. Additional components include a measurement circuit 608, an energy storage module 610, and an output circuit block 612. Processor 606 includes a charger control module 614, and RRD module 616, and advice module 617, and an optional capacitor reliability module 618. System 600 provides electrical shocks 620 to therapy electrodes 622 and 624, which are in contact with a patient 626. A sensor port 619 coupled to ECG sensing electrodes 609 is configured to send ECG signals generated by the ECG sensing electrodes to measurement circuit 608.

The functional blocks shown with a white background in FIG. 6 represent hardware that exists in some existing WCDs that employ conventional charger and energy storage components, such as a capacitor having an associated voltage rating. The functional blocks with a gray background (charger control module 614, advice module 617, and optional charge reliability module 618) in processor 606 represent new and/or modified modules to control the existing hardware to generate multi-stage charging in accordance with embodiments of the present disclosure. In some embodiments, the multi-stage charging function is implemented using both hardware (e.g., of the processor device) and the software of the charge control module 614. In other embodiments, the functionality of one or more of charger control module 614, RRD module 616, advice module 617, and optional charge reliability module 618 are implemented using embedded logic, such as predefined logic (e.g., one or more Application Specific Circuits (ASICs) and/or programmable logic (e.g., logic programmed into a Field Programmable Gate Array (FPGA), a (complex) programmable logic device (PLD or CPLD) and the like). As further illustrated, all or a portion of the functionality provided by RRD module 616 may be implemented in measurement circuit 608.

In some embodiments, the RRD module is configured to enable system 600 to monitor the heart rhythm of a patient to determine when shock therapy should be applied to the patient through one or more electrical shocks. For example, RRD module 616 may employ signals measured and/or output by measurement circuit 608 (alone or in combination with other circuitry not shown in FIG. 6) to detect heart arrhythmias using the techniques disclosed in one or more of U.S. Patent Publication No. 2021/0052180 (“Cardiac monitoring system with supraventricular tachycardia (svt) classifications”), and U.S. Pat. No. 9,592,403 (“Wearable cardioverter defibrillator (WCD) system making shock/no shock determinations from multiple patient parameters”), U.S. Pat. No. 9,757,581 (Wearable cardioverter defibrillator components making aggregate shock/no shock determination from two or more ECG signals), U.S. Pat. No. 11,058,885 (“Wearable cardioverter defibrillator (WCD) system detecting ventricular tachycardia and/or ventricular fibrillation using variable heart rate decision threshold”), U.S. Pat. No. 11,278,731 (“Wearable cardioverter defibrillator (WCD) system informing patient that it will not shock responsive to just-self-terminated cardiac arrhythmia”), and U.S. Pat. No. 11,471,693 (“Wearable cardioverter defibrillator (WCD) system choosing to consider ECG signals from different channels per QRS complex widths of the ECG signals”), each of which is incorporated by reference in its entirety for all purposes.

The circuitry shown in FIG. 6 is implemented in a WCD that provides defibrillation shocks in a manner like that described above. Battery 602 stores energy that is used by charger 604 to charge one or more capacitors in energy storage module 610 in preparation for delivering a defibrillation shock, as well as the other functions performed by the WCD. As above, battery 602 may be a rechargeable battery, a non-rechargeable battery, or a combination of rechargeable and non-rechargeable batteries.

Output circuit block 612 is illustrated as a switch for simplicity. However, output circuit block 612 can be implemented as an H-bridge in some embodiments. In addition, output circuit block 612 can include a relay or “main switch” (not shown) that can close to allow (or open to prevent) charge flowing through the output circuit to patient 626.

System 600 is configured to detect shockable arrhythmias and determine if one or more other criteria are met. In response to an arrhythmia detection and meeting such other criteria, processor 606 is configured to control output circuitry to allow charge stored in energy storage module 610 to flow through to therapy electrodes 622 and 624, thereby delivering a defibrillation shock to patient 626. Processor 606 may also provide input to energy storage module to configure circuitry in the module and/or selectively activate a dump circuit in the module (not separately shown).

Generally, advice module 617 supports the functionality provided by advice module 534 discussed above plus additional new functionality used to determine when to charge energy storage module 610, what charge level to be used, and when to dump charge stored in the energy storage module. While charge control module 614, RRD module 616, and advice module 617 are depicted as separate blocks in system 600, it will be recognized by those skilled in the art that this demarcation is exemplary and non-limiting, as similar functionality may be implemented using one or more software modules and/or one or more blocks of preprogrammed or programmable logic.

### Multi-Stage Charging of WCD Energy Storage Capacitor

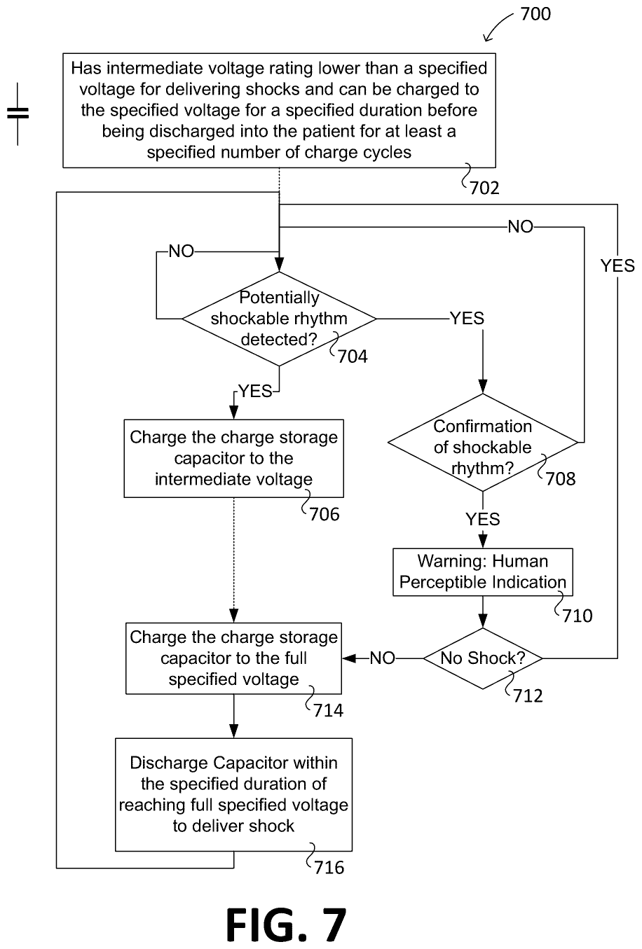

The size of a capacitor of a given type and capacitance typically increases with voltage rating. In accordance with embodiments of the present disclosure, methods of specifying and charging a WCD energy storage capacitor to enable the use of capacitors having lower continuous voltage ratings are provided. For example, FIG. 7 shows a flowchart 700 illustrating an exemplary process to deliver shocks to an ambulatory patient wearing a WDC having such an energy storage capacitor.

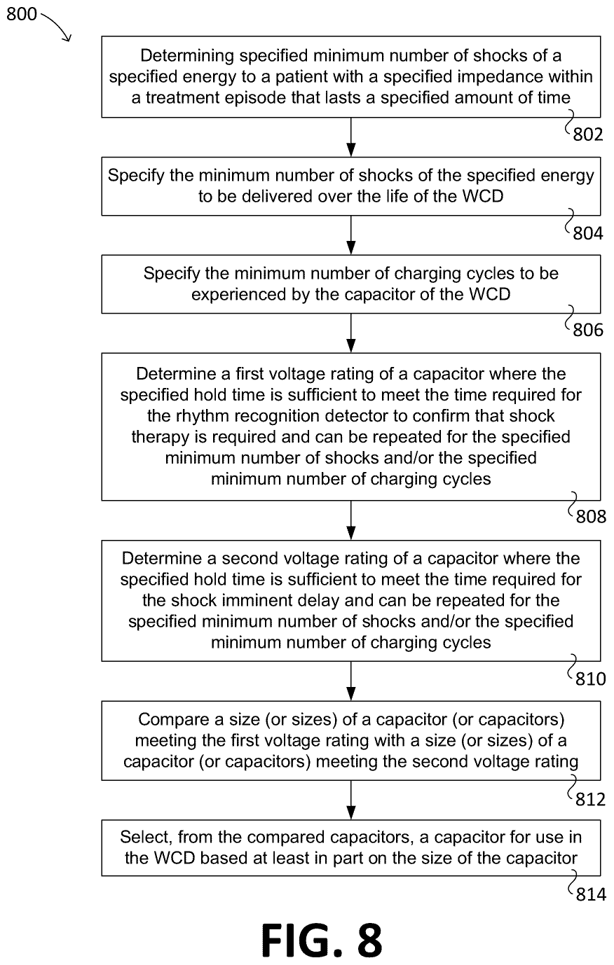

In block 702 of FIG. 7 an energy storage capacitor is provided in the WDC having an intermediate voltage rating that is lower than a specified voltage to which the capacitor is charged to deliver shocks and can be charged to the specified voltage for a specified duration before being discharged into the patient for at least a specified number of cycles. A process for specifying the energy storage capacitor is discussed below with reference to FIG. 8.

During operation of the WCD (i.e., when worn by a patient and activated), RRD functionality provided by RRD module and associated circuitry/components is used to detect heart rhythms that are indicative of potentially and confirmed shockable conditions. In flowchart 700 these are respectively referred to as potentially shockable rhythms and confirmed shockable rhythms in decision block 704 and 708. As mentioned above, in some embodiments the techniques disclosed in U.S. Pat. No. 11,364,388 are used to detect potentially shockable rhythms (that are associated with certain heart arrhythmias) and confirm the shockable rhythms.

In decision block 704 a determination is made to whether a potentially shockable rhythm has been detected. As depicted by the NO loopback, this determination is performed on an ongoing basis. In response to detection of a potentially shockable rhythm, the logic proceeds to block 706 to charge the energy storage capacitor to the intermediate voltage. As mentioned above, this intermediate voltage is less than the rated voltage for the capacitor. In decision block 708 a determination is made to whether the shockable rhythm is confirmed (i.e., validated), according to a validation criterion, meaning depending on whether or not the validation criterion is met. For example, the validation criterion may include that the detected cardiac arrhythmia (potential shockable rhythm) needs to be maintained for a threshold validation time. The determination of whether or not the detected shockable rhythm meets the validation criterion can be made from one or more physiological inputs (e.g., EEG sensor signals).

In some embodiments the timeframe for the operations of block 706 and decision block 708 will overlap. For example, a time period for the validation criterion may be started upon detection of a potentially shockable rhythm in decision block 704, with the energy storage capacitor being charged to the intermediate voltage during a portion of the time period.

When the shockable rhythm is not confirmed, the process flow returns to decision block 704. Generally, the charge in an energy storage capacitor may leak over time, with the leakage rate being somewhat variable amongst different capacitors. Thus, when the shockable rhythm is not confirmed, the charge in the energy storage capacitor may naturally dissipate to a voltage that is below the intermediate voltage.

When the shockable rhythm is confirmed in decision block 708, the answer is YES and the process proceeds to block 710 in which a warning comprising a human perceptible indication is provided to let the patient know that a shock is imminent. The patient is provided with an option to disable the shock within a warning period using one or more predetermined inputs, such as by activating a cancel switch on the WCD or other type of user input to effect cancellation of a pending shock in the manner discussed above. As depicted in decision block 712, if the patient activates the cancel switch (or other means to effect cancellation) a determination there should be no shock results (answer is YES), and the process returns to decision block 704 to begin a next cycle. If a cancellation input is not received (e.g., within a predefined cancellation period), the answer to decision block 714 is NO and the flow proceeds to block 714 in which the energy storage capacitor is charged to the full specified voltage to be used for delivering a shock. The capacitor is then discharged within the specified duration of reaching the full specified voltage to deliver a shock to the patient, as shown in block 716. The process then loops back to decision block 704 to begin the next cycle.

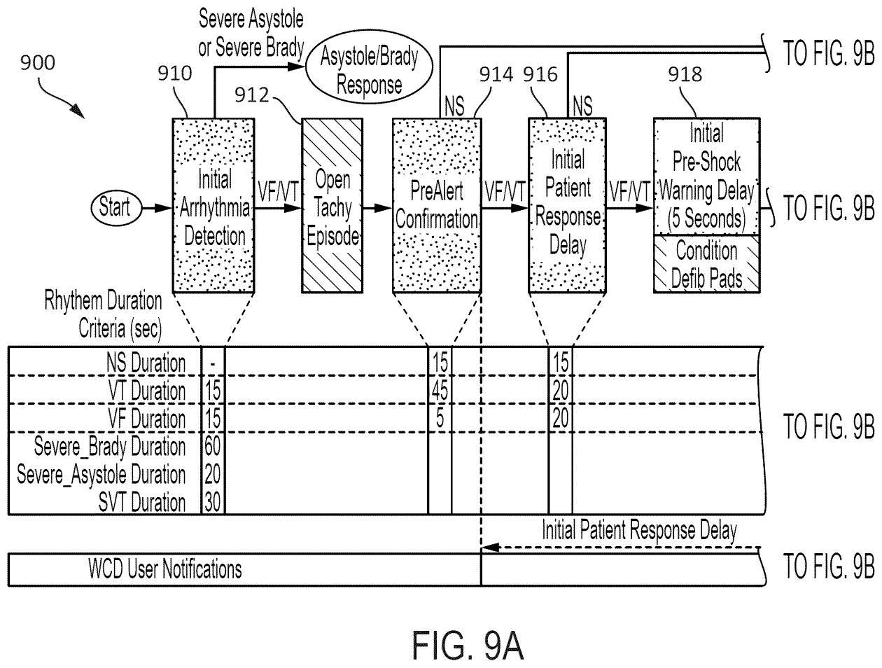

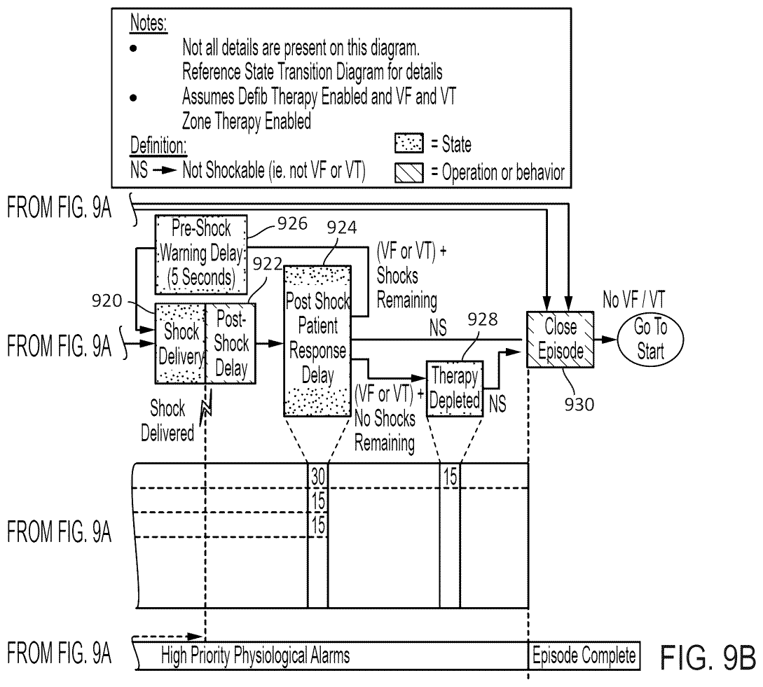

Generally, the confirmation time (time period during which a shockable rhythm is confirmed) may vary, depending on the type of shockable rhythm and other considerations. For example, an extended confirmation time may be employed under some embodiments. An example of an extended confirmation time for ventricular tachycardia (VT) in accordance with one or more embodiments is illustrated in FIGS. 9A and 9B and discussed below.

### Determining Energy Storage Capacitor Health

In some embodiments, the health of the energy storage capacitor is determined using charge reliability module 618. In one embodiment, charge reliability module maintains a count of the number of instances when the energy storage capacitor is charged to the full specified voltage. The charge reliability module uses a health model that calculates a health score based on a function of the count. The charge reliability module also tracks an aggregated time when the energy storage capacitor is charged at the full specified voltage, and employs a health model based on the aggregated time. Under another embodiment, the health model is based on a combination of the count and the aggregated time.

Another reliability factor to monitor is the time it takes to charge the capacitor. As the capacitor's health declines and it becomes more leaky, it will take longer to charge to voltage. This can also be affected by battery voltage so that may need to be considered. Another test of the capacitor could be performed after charging to the intermediate voltage by monitoring the voltage after the charger is turned off for some time period to see how fast it self discharges. In some embodiments, one or both of these monitoring operations may be employed.

Generally, the health models may be established using one or more mathematical formulas that are derived using projected and/or historical data. For example, such models may be derived from statistical analysis of energy storage capacitor reliability based on test samples and/or actual in field usage data. Under one approach, test sample data and/or field usage data is provided as inputs to a train a machine learning model that is implemented with charge reliability module 618. For example, such machine learning models may be implemented in software that is executed on processor 606 or using preprogrammed logic (e.g., an ASIC) or programable logic (e.g., in an FPGA).

### Example Embodiments of Methods to Specify a WCD Energy Storage Capacitor

The following embodiments are directed to determining or selecting an energy storage capacitor to store charge in a WCD using the foregoing multi-stage charging solutions. One process flow showing exemplary considerations and decisions is shown in a flowchart 800 in FIG. 8.

The process begins in block 802 by determining or identifying the specified minimum number of shocks of a specified energy to a patient with a specified impedance within a treatment episode that lasts a specified amount of time. In one embodiment the specified energy is 170 Joule. In one embodiment, the WCD delivers up to 5 shocks in one episode to convert a patient's rhythm, and if it doesn't convert after 5 shocks a call is made to 911. In a maximum case scenario, the rhythm would be converted on the 5^{th} shock. This could be repeated for 5 episodes, in one embodiment, with the result being the capacitor is designed to withstand 25 shocks in a short period of time.

In block 804 the minimum number of shocks of the specified energy to be delivered over the life of the WCD is specified, while the minimum number of charging cycles to the full specified voltage to be experienced by the capacitor of the WCD is specified in block 806.

In block 808 a first voltage rating of a capacitor is determined where the specified hold time is sufficient to meet the time required for the rhythm recognition detector to confirm that shock therapy is required and can be repeated for the specified minimum number of shocks and/or the specified minimum number of charging cycles. In block 810 a second voltage rating of a capacitor is determined where the specified hold time is sufficient to meet the time required for the shock imminent delay and can be repeated for the specified minimum number of shocks and/or the specified minimum number of charging cycles.

Next, in block 812, a size (or sizes) of a capacitor (or capacitors) meeting the first voltage rating is compared with a size (or sizes) of a capacitor (or capacitors) meeting the second voltage rating. In block 814 a capacitor for use in the WCD is selected from the compared capacitors based at least in part on the size of the capacitor.

### Extended Confirmation Time for VT

FIGS. 9A and 9B collectively show a diagram illustrating an extended confirmation time for VT in accordance with one or more embodiments. In method 900, arrhythmia detection starts with a 15 second initial arrhythmia detection period at block 910. If the rhythm is detected as being shockable for 15 seconds, then an episode is opened at block 912. After an episode is opened, there is a pre-alert confirmation period at block 914 before an alarm is given. In some embodiments, for VF the confirmation period at block 914 is five seconds and for VT the confirmation period at block 914 is 45 seconds. In other embodiments, different confirmation periods may be utilized. The WCD alarms for a period of time referred to as the Initial Patient Response Delay at block 916. If the patient does not respond, then a shock is given at block 920 after an Initial Pre-Shock Warning Delay at block 918, which may be, for example, five seconds. A Post Shock Delay at block 922 occurs after shock delivery, followed by a Post Shock Patient Response Delay at block 924. Depending on the state of VF or VT and whether there are shocks remaining to be delivered by WCD 100, another shock delivery may occur at block 920 after Pre-Shock Warning Delay at block 926. The amount of energy stored in the battery can be sufficient to deliver one or more shocks during a given episode until the energy stored in the battery is depleted. After a first shock, if there is enough energy in the battery, the energy storage capacitor can be recharged one or more times to deliver one or more additional shocks. In some embodiments, the energy storage capacitor is recharged after every shock, and it typically takes about six seconds to charge the energy storage capacitor for each shock. Application of multiple shocks can occur via a loop comprising block 920, block 922, block 924, and block 926. This loop can be repeated depending on the state of VF or VT and whether there are any remaining shocks to be delivered based on the amount of energy remaining in the battery. If therapy is depleted at block 928, meaning there is not enough energy left in the battery to deliver another shock or a maximum number of unsuccessful shocks have been delivered. If there is no longer VF or VT, the episode can be closed at block 930.

Although some embodiments have been described in reference to particular implementations, other implementations are possible according to some embodiments. Additionally, the arrangement and/or order of elements or other features illustrated in the drawings and/or described herein need not be arranged in the particular way illustrated and described. Many other arrangements are possible according to some embodiments.

The circuitry and associated circuit elements illustrated and described herein are exemplary and non-limiting. Those having skill in the art with recognize components made by the same or other vendors may be implemented in place of the components illustrated in the Figures and that the values for circuit elements such as resistor, capacitor, switches, and voltages are exemplary and non-limiting.

In each system shown in a Figure, the elements in some cases may each have the same reference number or a different reference number to suggest that the elements represented could be different and/or similar. However, an element may be flexible enough to have different implementations and work with some or all of the systems shown or described herein. The various elements shown in the figures may be the same or different. Which one is referred to as a first element and which is called a second element is arbitrary.

In the description and claims, the terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still co-operate or interact with each other. Additionally, “communicatively coupled” means that two or more elements that may or may not be in direct contact with each other, are enabled to communicate with each other. For example, if component A is connected to component B, which in turn is connected to component C, component A may be communicatively coupled to component C using component B as an intermediary component.

An embodiment is an implementation or example of the inventions. Reference in the specification to “an embodiment,” “one embodiment,” “some embodiments,” or “other embodiments” means that a particular feature, structure, or characteristic described in connection with the embodiments is included in at least some embodiments, but not necessarily all embodiments, of the inventions. The various appearances “an embodiment,” “one embodiment,” or “some embodiments” are not necessarily all referring to the same embodiments.

Not all components, features, structures, characteristics, etc. described and illustrated herein need be included in a particular embodiment or embodiments. If the specification states a component, feature, structure, or characteristic “may”, “might”, “can” or “could” be included, for example, that particular component, feature, structure, or characteristic is not required to be included. If the specification or claim refers to “a” or “an” element, that does not mean there is only one of the element. If the specification or claims refer to “an additional” element, that does not preclude there being more than one of the additional elements.

An algorithm is here, and generally, considered to be a self-consistent sequence of acts or operations leading to a desired result. These include physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers or the like. It should be understood, however, that all of these and similar terms are to be associated with the appropriate physical quantities and are merely convenient labels applied to these quantities.

As discussed above, various aspects of the embodiments herein may be facilitated by corresponding software and/or firmware components and applications, such as software and/or firmware executed by an embedded processor or the like. Thus, embodiments of this invention may be used as or to support a software program, software modules, firmware, and/or distributed software executed upon some form of processor, processing core or embedded logic a virtual machine running on a processor or core or otherwise implemented or realized upon or within a non-transitory computer-readable or machine-readable storage medium. A non-transitory computer-readable or machine-readable storage medium includes any mechanism for storing or transmitting information in a form readable by a machine (e.g., a computer). For example, a non-transitory computer-readable or machine-readable storage medium includes any mechanism that provides (i.e., stores and/or transmits) information in a form accessible by a computer or computing machine (e.g., computing device, electronic system, etc.), such as recordable/non-recordable media (e.g., read only memory (ROM), random access memory (RAM), magnetic disk storage media, optical storage media, flash memory devices, etc.). The content may be directly executable (“object” or “executable” form), source code, or difference code (“delta” or “patch” code). A non-transitory computer-readable or machine-readable storage medium may also include a storage or database from which content can be downloaded. The non-transitory computer-readable or machine-readable storage medium may also include a device or product having content stored thereon at a time of sale or delivery. Thus, delivering a device with stored content, or offering content for download over a communication medium may be understood as providing an article of manufacture comprising a non-transitory computer-readable or machine-readable storage medium with such content described herein.

The operations and functions performed by various components described herein may be implemented by software running on a processing element, via embedded hardware or the like, or any combination of hardware and software. Such components may be implemented as software modules, hardware modules, special-purpose hardware (e.g., application specific hardware, ASICs, DSPs, etc.), embedded controllers, hardwired circuitry, hardware logic, etc. Software content (e.g., data, instructions, configuration information, etc.) may be provided via an article of manufacture including non-transitory computer-readable or machine-readable storage medium, which provides content that represents instructions that can be executed. The content may result in a computer performing various functions/operations described herein.

As used herein, a list of items joined by the term “at least one of” can mean any combination of the listed terms. For example, the phrase “at least one of A, B or C” can mean A: B: C: A and B: A and C: B and C: or A, B and C.

The above description of illustrated embodiments of the invention, including what is described in the Abstract, is not intended to be exhaustive or to limit the invention to the precise forms disclosed. While specific embodiments of, and examples for, the invention are described herein for illustrative purposes, various equivalent modifications are possible within the scope of the invention, as those skilled in the relevant art will recognize.

These modifications can be made to the invention in light of the above detailed description. The terms used in the following claims should not be construed to limit the invention to the specific embodiments disclosed in the specification and the drawings. Rather, the scope of the invention is to be determined entirely by the following claims, which are to be construed in accordance with established doctrines of claim interpretation.

1. A wearable cardioverter defibrillator (WCD) for an ambulatory patient, comprising:
a support structure configured to be worn by the ambulatory patient;
WCD circuitry, operatively coupled to the support structure, including,
a power source;
a charger, coupled to the power source;
an energy storage module, operatively coupled to the charger, including an energy storage capacitor having a rated voltage;
output circuitry, coupled to the energy storage module; and
control circuitry operatively coupled to the charger, the energy storage module, and the output circuitry, the control circuitry configured to implement a multi-stage charging scheme under which the energy storage capacitor is charged to a first intermediate voltage below its rated voltage during a first charging stage and charged to a second voltage above the rated voltage during a second charging stage; and
first and second therapy electrodes coupled to the output circuitry and configured to be maintained on a body of the ambulatory patient when the support structure is worn by the ambulatory patient;

2. The WCD of claim 1, further comprising:
at least one sensor to sense a parameter of the ambulatory patient; and
a measurement circuit, operatively coupled to the at least one sensor, configured to output one or more signals in responsive to the sensed parameter,
wherein the WCD circuitry is configured, responsive to the one or more output signals, to detect heart arrhythmias that may be treated with shocks and control when the shocks are delivered to the first and second therapy electrodes, and wherein the energy storage capacitor is charged to the second voltage prior to delivering a shock.

3. The WCD of claim 2, wherein the WCD circuitry is configured to:
detect a potentially shockable rhythm; and
charge the energy storage capacitor to the intermediate voltage in response to detection of a potentially shockable rhythm.

4. The WCD of claim 3, wherein the WCD circuitry is further configured to:
confirm the shockable rhythm; and
provide a warning comprising a human perceptible indication that delivery of a shock is imminent.

5. The WCD of claim 4, further comprises means for enabling the ambulatory patient to provide a cancellation input or signal to cancel a shock, and wherein the WCD circuitry is further configured to:
determine that a cancellation input or signal has not been detected or received within a cancellation period following the warning;
charge the energy storage capacitor to the second voltage; and
discharge the energy storage capacitor to deliver a shock to the ambulatory patient via the first and second therapy electrodes.

6. The WCD of claim 3, wherein the WCD circuitry is further configured to.
confirm the shockable rhythm;
charge the energy storage capacitor to the second voltage; and
discharge the energy storage capacitor to deliver a shock to the ambulatory patient via the first and second therapy electrodes within a specified duration of reaching the second voltage.

7. The WCD of claim 3, wherein the WCD circuitry is further configured to:
in response to confirmation of the shockable rhythm, provide a warning comprising a human perceptible indication that delivery of a defibrillator shock is imminent,
charge the energy storage capacitor to the second voltage;
determine when a cancellation input or signal has been detected or received to cancel the defibrillator shock; and
in response to thereto,
not deliver a shock; and
dump charge in the energy storage capacitor to reduce the voltage below its rated voltage.

8. The WCD of claim 2, further comprising a Rhythm Recognition Detector (RRD) module configured to detect potential shockable rhythms and confirm shockable rhythms by processing one or more signals output by the measurement circuit.

9. The WCD of claim 2, wherein the control circuitry includes:
one or more processors; and
one or more software modules comprising instructions configured to be executed on at least one of the one or more processors to,
control the charger to implement the two-stage charging scheme in response to detection of shockable rhythms; and
control delivery of shocks to the ambulatory patient.

10. The WCD of claim 2, wherein the WCD circuitry is further confirmed to:
maintain at least one of (a) a count of a number of times the charge storage capacitor is charged to the second voltage and (b) an aggregate amount of time the charge storage capacitor is charged at the second voltage; and
determine a health of the energy storage capacitor as a function of at least one of a) and b).

11. An apparatus, including circuitry comprising:
an energy storage module including an energy storage capacitor having a rated voltage;
a charger, configured to be coupled to a battery and provide a charge to the energy storage module,
output circuitry, coupled to the energy storage module; and
control circuitry operatively coupled to the charger, the energy storage module, and the output circuitry, the control circuitry configured to implement a multi-stage charging scheme under which the energy storage capacitor is charged to a first intermediate voltage below its rated voltage during a first charging stage and charged to a second voltage above the rated voltage during a second charging stage.

12. The apparatus of claim 11, wherein the apparatus is configured to be implemented in a wearable cardioverter defibrillator (WCD) for an ambulatory patient, the WCD including first and second therapy electrodes and at least one sensor configured to sense a parameter of the ambulatory patient, the apparatus further comprising:
a measurement circuit, operatively coupled to the at least one sensor, configured to output one or more signals in responsive to the sensed parameter,
wherein the apparatus is configured, responsive to the one or more output signals, to detect heart arrhythmias that may be treated with shocks and control when the shocks are delivered to the first and second therapy electrodes, and wherein the energy storage capacitor is charged to the second voltage prior to delivering a shock.

13. The apparatus of claim 12, further configured to:
detect a potentially shockable rhythm; and
charge the energy storage capacitor to the intermediate voltage in response to detection of a potentially shockable rhythm.

14. The apparatus of claim 12, further configured to:
confirm the shockable rhythm; and
provide a warning comprising a human perceptible indication that delivery of a shock is imminent.

15. The apparatus of claim 12, wherein the WCD includes means for enabling the ambulatory patient to provide a cancellation input or signal to cancel a shock that is operatively coupled to the apparatus, further configured to:
determine that a cancellation input or signal has not been detected or received within a cancellation period following the warning;
charge the energy storage capacitor to the second voltage; and
discharge the energy storage capacitor to deliver a shock to the ambulatory patient via the first and second therapy electrodes.

16. The apparatus of claim 13, further configured to.
confirm the shockable rhythm;
charge the energy storage capacitor to the second voltage; and
discharge the energy storage capacitor to deliver a shock to the ambulatory patient via the first and second therapy electrodes.

17. The apparatus of claim 12, further comprising a Rhythm Recognition Detector (RRD) module configured to detect potential shockable rhythms and confirm shockable rhythms by processing one or more signals output by the measurement circuit.

18. The apparatus of claim 12, wherein the control circuitry includes:
one or more processors; and
one or more software modules comprising instructions configured to be executed on at least one of the one or more processors to,
control the charger to implement the two-stage charging scheme in response to detection of shockable rhythms; and
control delivery of shocks to the ambulatory patient.

19. A method implemented by a wearable cardioverter defibrillator (WCD) worn by an ambulatory patient, the WCD including a battery coupled to WCD circuitry including a charger, an energy storage module including an energy storage capacitor having a rated voltage, control circuitry, and output circuitry coupled to first and second therapy electrodes in contact with the ambulatory patient, comprising:
implementing a multi-stage charging scheme under which the energy storage capacitor is charged to a first intermediate voltage below its rated voltage during a first charging stage and charged to a second voltage above the rated voltage during a second charging stage.

20. The method of claim 19, further comprising
detecting heart arrhythmias that may be treated with shocks and controlling when the shocks are delivered to the ambulatory patient via the first and second therapy electrodes, wherein the energy storage capacitor is charged to the second voltage prior to delivering a shock.

21. The method of claim 20, further comprising:
detecting a potentially shockable rhythm; and
charging the energy storage capacitor to the intermediate voltage in response to detection of a potentially shockable rhythm.

22. The method of claim 21, further comprising:
confirming the shockable rhythm;
providing a warning comprising a human perceptible indication that delivery of a shock is imminent;
determining that a cancellation input or signal has not been detected or received within a cancellation period following the warning;
charging the energy storage capacitor to the second voltage; and
discharging the energy storage capacitor to deliver a shock to the ambulatory patient via the first and second therapy electrodes.

23. The method of claim 21, further comprising:
confirming the shockable rhythm;
charging the energy storage capacitor to the second voltage; and
discharging the energy storage capacitor to deliver a shock to the ambulatory patient via the first and second therapy electrodes within a specified duration of reaching the second voltage.

24. The WCD of claim 21, further comprising:
confirming the shockable rhythm;
provide a warning comprising a human perceptible indication that delivery of a defibrillator shock is imminent,
charging the energy storage capacitor to the second voltage;
determining a cancellation input or signal has been detected or received to cancel the defibrillator shock; and
in response to thereto,
not delivering a shock; and
dumping charge in the energy storage capacitor to reduce the voltage below its rated voltage.

25. The method of claim 19, further comprising:
maintaining at least one of (a) a count of a number of times the energy storage capacitor is charged to the second voltage and (b) an aggregate amount of time the energy storage capacitor is charged at the second voltage; and
determining a health of the energy storage capacitor as a function of at least one of a) and b).
